(19) United States
(12) Patent Application Publication
(10) Pub. No.: US 2020/0141691 A1
(43) Pub. Date: May 7, 2020

(54) GUN STOCK HAND WARMER

(71) Applicant: Gregory Wayne Atkinson, Statesville, NC (US)

(72) Inventor: Gregory Wayne Atkinson, Statesville, NC (US)

(21) Appl. No.: 16/546,030

(22) Filed: Aug. 20, 2019

Related U.S. Application Data
(60) Provisional application No. 62/720,731, filed on Aug. 21, 2018.

(51) Int. Cl. F41C 27/00; A61F 7/03

(52) U.S. Cl. CPC F41C 27/00; A61F 2007/0036; A61F 7/03

(57) ABSTRACT

A gun stock hand warmer for use with a long gun having a stock with a butt and a receiver with a trigger guard. The hand warmer has a first tubular body shaped and dimensioned to enclose the butt of the gun, a second to body shaped and dimensioned to enclose the receiver and trigger guard, and a third of the gun. The first and second tubular bodies are in overlapping relationship and share a common interior wall.

### FIELD OF HE INVENTION

The present invention relates gun stock sleeves. More particularly the present invention relates to gun stock sleeves which can warm or protect the hand of the shooter.

### BACKGROUND OF THE INVENTION

Gun stock sleeves are known for protecting the stock of the gun and, in some cases, having a feature for warming and/or protecting the hand that will be used to operate the trigger of the gun. For example, U.S. Pat. No. 4,947,573, Aug. 14, 1990 to Eric Bischoff for GUN STOCK MITTEN relates to a gun stock sleeve with a mitten for use in adverse weather conditions by persons carrying rifles, shotguns or like firearms or guns having a stock. Another example is U.S. Pat. No. 4,754,498 to James L. Stinemates for GUN MITTEN which relates to a gun mitten having a body formed of flexible material which may be quilted or otherwise enhanced with insulating material to provide a substantially weatherproof enclosure for the stock, grip, trigger and trigger guard area of the gun while also enclosing the shooters hand and forearm.

Although gun stock sleeves are known in the prior art, there remains room for an improved gun stock sleeve which can be economically manufactured and which offers protection and warmth to the hand of the user. Accordingly, the present invention relates to an improved gun stock sleeve which can be readily and economically manufactured and which is referred to herein as a “gun stock hand warmer.” The gun stock hand warmer of this invention can warm and protect the hand that will be used to operate the trigger of the gun while allowing access to the trigger of the gun as well as easy and quiet removal of the hand from the hand warmer. The gun stock hand warmer of the present invention has several advantageous features. It can be made of waterproof material and protects and warms the shooting hand. It can carry an exo-thermal hand warming device to provide additional heat. It allows the hand of the shooter to operate the trigger of the gun without removing the hand from the sleeve. It fits right or left handed shooters and long guns including rifles and shotguns with or without scopes. It protects the stock from scratches and can carry a recoil pad which can be permanently attached to the gun stock hand warmer or removable therefrom and can be used instead of the recoil pad on the long gun or in addition to a recoil pad on the long gun. It is made of separate panels which readily fabricated and then sewn or otherwise attached together in an economical manufacturing process. The gun stock hand warmer is easily installed on a long gun and is easily moved from one gun to another.

Of course, even further advantageous features of the present invention may become apparent to those skilled in the art. For example, a hunter using a gun stock hand warmer of this invention may greatly appreciate that the hunter's hand can be quietly withdrawn from the sleeve or moved into shooting position without making noise which might disturb nearby game. Also, the gun stock hand warmer can have a pair of loops for attachment of a strap of a rifle or the like. Thus, when the gun stock hand warmer is used by a right handed shooter, a loop is provided at the bottom of the hand warmer for attachment of a strap. And when the gun stock hand warmer is used by a left handed shooter, the hand warmer is rotated 180 degrees to allow insertion of the shooters left hand and a strap is provided at the bottom of the now rotated hand warmer for attachment of a strap.

Further understanding of the present invention will be had from the following description and claims taken in conjunction with the accompanying drawings.

### BRIEF DESCRIPTION O HE DRAWINGS

FIG. 1 is a side elevation of a preferred embodiment of the present invention shown in use with a rifle with user and rifle broken away.

FIG. 2 a side elevation of the preferred embodiment of FIG. 1 shown in combination with a rifle broken away;

FIG. 3 is a side elevation of the back panel of the embodiment of FIG. 1;

FIG. 4 is a side elevation of the intermediate panel of the embodiment of FIG. 1.

FIG. 5 is a side elevation of the front panel of the embodiment of FIG. 1:

FIG. 6 is a side elevation of the embodiment of FIG. 1;

FIG. 7 is an end view taken in the direction of arrow 7 in FIG. 6; and

FIG. 8 is a sectional view taken along line 8-8 in FIG. 8.

### SUMMARY OF THE INVENTION

A gun stock hand warmer has back, intermediate and front panels which are joined as described herein to provide a first tubular body shaped and dimensioned to enclose the butt of the gun, a second tubular body shaped and dimensioned to enclose the receiver, trigger and trigger guard of the gun, and a third tubular body for receiving the hand of a user. The gun stock hand warmer has an intermediate portion where the first and third tubular bodies share a common panel, the forward portion of the intermediate panel. The first and second tubular bodies have open ends allowing their placement over an associated gun. The third tubular body has an open rear end to allow entry of the hand of the user for warming the hand and an open forward end allowing the hand to be positioned adjacent the trigger guard of the gun. The hand warmer is designed to be easily and economically manufactured and yet also to be effective for its intended purpose in warming the hand of a hunter or the like while allowing the hand of the hunter to readily access the trigger of the gun or to be withdrawn from the hand warmer.

### DESCRIPTION OF THE INVENTION

The present invention relates to a gun stock hand warmer for a long gun having a butt with a grip and a receiver with a trigger and a trigger guard. Broadly speaking, the gun stock hand warmer comprises three flexible panels which are sewn or otherwise secured to each other to provide first and second tubular bodies as described in more detail below.

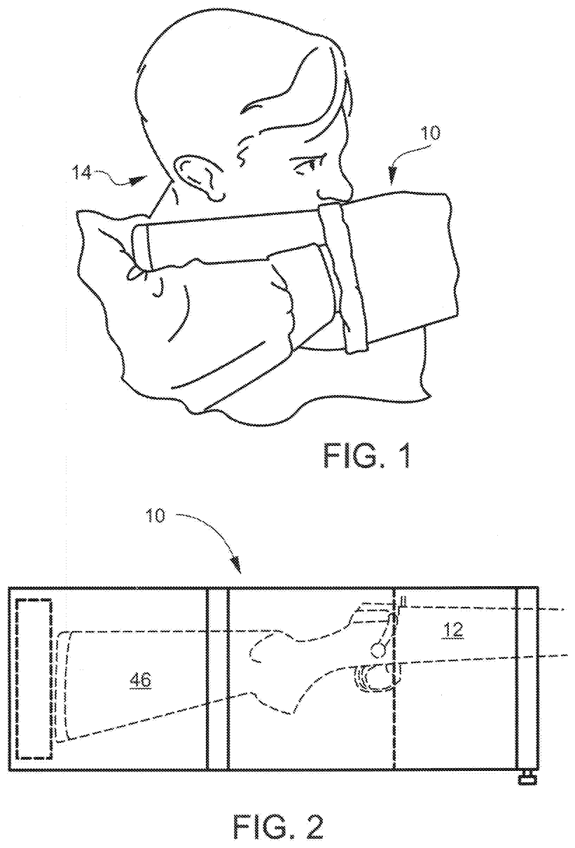

Now referring to FIG. 1, a preferred embodiment of a gun stock hand warmer of the present invention shown and indicated by the numeral 10. Gun stock hand w warmer 10 is shown installed on a rifle 12 and in use with by a person 14.

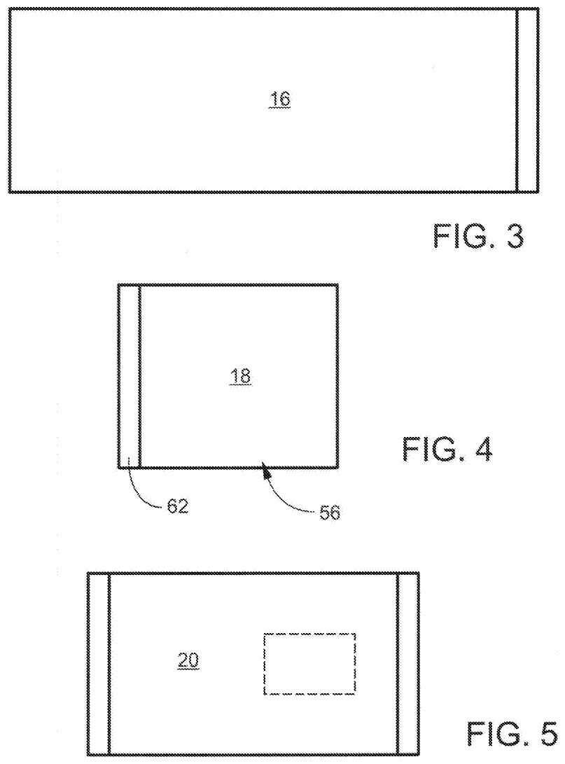

As shown in FIGS. 2-8, gun stock hand warmer 10 comprises back panel 16, intermediate panel 18, and front panel 20. It is contemplated that each panel 16, 18, and 20 will be comprised of flexible textile material such as cotton, synthetic cloth or the like and preferably such material will be waterproof and wind resistant. For some uses, the material may be lined with insulating material for further warmth.

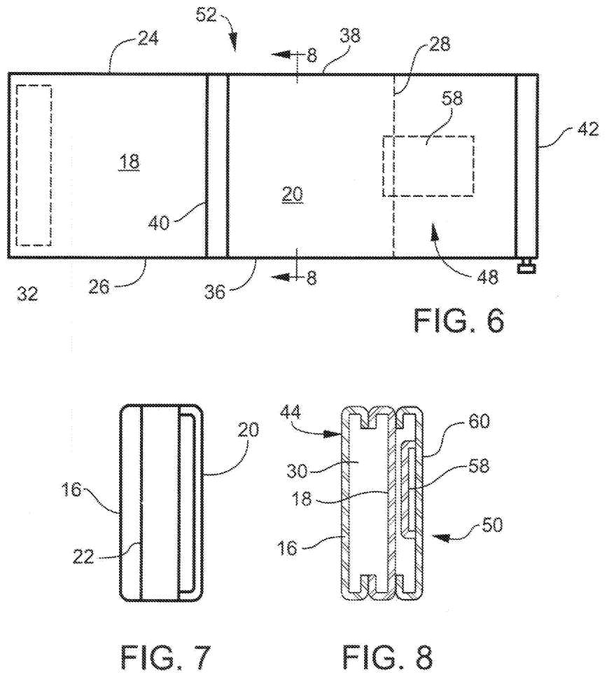

As best shown in FIGS. 6-8, intermediate panel 18 is joined to back panel 16 along edges 22, 24 and 26 by sewing or other suitable means. Intermediate panel 18 does not extend the length of back panel 1$ and its forward end 28 is unattached thus providing an opening for receiving the butt of an associated long gun in space 30 between intermediate panel 18 and back panel 16. A recoil pad 32 may optionally be positioned at the rearward end portion 34 between intermediate panel 18 and back panel 16.

Sides 36 and 38 of front panel 20 are sewn or otherwise attached to back panel 16 along the length of front panel 20. Both rearward end 40 and forward end 42 of front panel 20 are left unsecured.

As thus constructed, stock hand warmer 10 comprises a first tubular body 44 shaped and dimensioned to enclose the butt 46 of rifle 12, a second tubular body 48 shaped and dimensioned to enclose the receiver and trigger guard of rifle 12, and a third tubular body 50 shaped and dimensioned to receive the hand and wrist of a user. Gun stock hand warmer 10 has an intermediate portion 52 where the first and third tubular bodies, 44 and 50 share a common panel, a forward portion of intermediate panel 18 indicated at 56.

Optionally, a pocket 58 can be provided in second tubular body 48 for storage of an exothermic packet 60 or other exothermic device to provide heat for warming the hand of the user.

Elastic material 62 can be sewn at the rearward end of front panel 18 to provide a snug fit around the arm or wrist of the user. A draw string 64 can be attached to the forward end of gun stock hand warmer 10 to for secure, placement about rifle 12.

Loops 66 and 68 are preferably provided for use with a sling or strap of an associated long gun. Loop 66 is for attachment of a sling for use with a right handed shooter while loop 68 is for attachment of a sling when the gun stock hand warmer of the present invention is used by a left handed shooter.

It will be appreciated by hose skilled in the art that a gun stock hand warmer of the present invention should be made of high quality materials such waterproof textiles or the like. Also, it will be appreciated by those skilled in the art that the present invention is subject to modification and variation. It is intended that such modifications and variations are considered to be within the broad scope of the invention which is intended to be limited only by the following claims.

What is claimed is:

1. A gun stock hand warmer for use by a person's hand and a long gun having a stock with a butt and a receiver with a trigger guard, the gun stock hand warmer comprising a first tubular body shaped and dimensioned to enclose the butt, a second tubular body shaped and dimensioned to enclose the receiver and trigger guard, and a third tubular body shaped and dimensioned to receive the hand of the user, said gun stock hand warmer having an intermediate portion where said first and third tubular bodies share a common wall.

2. A gun stock hand warmer as in claim 1 wherein said second body has a forward end portion configured to fit snugly around the gun forward of the trigger guard.

3. A gun stock hand warmer as in claim 2 wherein said first body has a recoil pad positioned at rearward portion thereof.

4. A gun stock hand warmer as in claim 2 wherein said second body has a pocket dimensioned to hold an exo-thermal hand warming device.

5. A gun stock hand warmer as in claim 2 wherein said third body has an elastic band around a rearward end thereof.

6. A gun stock hand warmer as in claim 1 wherein a pair of loops are secured to said first tubular body for attachment of a sling thereto.
